(19) United States
(12) Patent Application Publication
(10) Pub. No.: US 2019/0183922 A1
(43) Pub. Date: Jun. 20, 2019

(54) NOVEL ACTIVE IMMUNOMODULATORY AGENT AND COMPOSITION CONTAINING SAME

(71) Applicants: UNIVERSITE CLERMONT AUVERGNE, CLERMONTFERRAND (FR); SIGMA CLERMONT, AUBIERE CEDEX (FR); CENTRE NATIONAL DE LA RECHERCHE SCIENTIFIQUE, PARIS (FR); UNIVERSITE DE LA ROCHELLE, LA ROCHELLE (FR); UNIVERSITE D'ANTSIRANANA, ANTSIRANANA (MG)

(72) Inventors: Cédric DELATTRE, BEAUMONT (FR); Philippe MICHAUD, BILLOM (FR); Guillaume PIERRE, CLERMONT-FERRAND (FR); Nicolas BRIDIAU, CIRE D'AUNIS (FR); Thierry MAUGARD, LA JARNE (FR); Taratra Andrée FENORADOSOA, ANTSIRANANA (MG); Hernas Martial RAKOTOARISOA, ANTSIRANANA (MG)

(21) Appl. No.: 16/330,017

(22) PCT Filed: Aug. 28, 2017
(86) PCT No.: PCT/EP2017/071492

§ 371 Date: Mar. 1, 2019

(30) Foreign Application Priority Data
Sep. 2, 2016 (FR) 1658201

(51) Int. Cl. A61K 31/737; A61K 36/04; A61Q 19/00; A61P 17/00; A61P 29/00; A61K 45/06; A61K 8/73

(52) U.S. Cl. CPC A61K 31/737; A61K 36/04; A61Q 19/00; A61K 8/73; A61P 29/00; A61K 45/06; A61P 17/00

(57) ABSTRACT

Disclosed is a sulphated polysaccharide extracted from a red alga of the Haliptilon subulatum species or a salt thereof as active ingredient. More particularly, the present application relates to a sulphated polysaccharide extracted from a red alga of the Haliptilon subulatum species or a salt thereof as active ingredient, for its use in the modulation of the immune response in humans or animals, in particular in the prevention and/or treatment of inflammatory diseases, in particular of the skin.

The present invention relates to a sulphated polysaccharide extracted from a red alga for use thereof in the modulation of the immune response in humans or animals.

Skin is a keratinized multi-stratified epithelium surrounding the entire outer surface of humans. In particular, skin acts as:
a mechanical barrier to the development of bacteria, viruses and parasites, due to a low permeability and the desquamation of the skin,
a chemical barrier having antimicrobial proteins and peptides which can lead to: mechanical rupture of the bacterial membranes, enzymatic destructuration of the bacterial membranes and nutrient sequestration,
a biological barrier having a commensal flora which is a set of bacteria located on the skin and the mucous membranes and playing an important role as a barrier.

Skin is in constant contact with numerous environmental factors (viruses, parasites and bacteria, but also exposure to the sun and UV in particular) that are likely to damage it. In response to various environmental factors, the immune system develops defence mechanisms that are natural (innate immunity, also called natural or non-specific immunity) and acquired (adaptive or specific immunity).

When the infectious agent has crossed the epidermal barrier, the molecular units characteristic of the micro-organisms will interact with the Toll-like receptors present on the membrane of the keratinocytes, dendritic cells, mastocytes and macrophages. These activated cells become effective (phagocytosis of the bacteria by the dentritic cells and the macrophages) and release vasodilating mediators allowing exudation of plasma and inflammatory cytokines such as TNFα, IL1α, IL8, which again amplifies the vasodilation and vasopermeabilization reaction. VEGF (vascular endothelial growth factor) is an epidermal factor essential to the vasodilation process. Its expression is often enhanced in pathologies exhibiting vascular anomalies, for example couperose, and the keratinocytes are capable of secreting VEGF under the effect of, among other things, the inflammatory cytokines. VEGF thus acts directly on the endothelial cells of the blood vessel walls and thus leads to a dilation and permeabilization of this wall. In addition, it promotes the production of enzymes called MMPs (Matrix Metalloproteases) which are capable of degrading the support fibres of the dermis (collagen and elastin); this degradation thus participates in the relaxation of the blood vessel walls and their weakening leading to permanent redness.

Creams comprising corticoid-, dermocorticoid-, anti-acne and oestrogen-type compounds or also steroid or non-steroid anti-inflammatories, obtained by chemical synthesis are commonly used to treat skin inflammations. However, the chemical compounds contained in these creams can produce side effects, such as allergic reactions in particular, when they are administered to the patient.

Consequently, it appears necessary to have novel compositions of natural origin that are capable of inhibiting or antagonizing the effects of the VEGF, in order to prevent and/or treat inflammatory diseases, in particular of the skin, while reducing the risks of side effects.

In a completely surprising manner, the inventors have shown that the sulphated polysaccharides extracted from the marine red alga of the Haliptilon subulatum species have useful immunomodulatory properties, in particular relating to the inhibition of the release of vascular endothelial growth factor (VEGF), by the cells involved in the inflammatory process and/or as VEGF antagonists.

Thus a subject of the present invention is a sulphated polysaccharide extracted from a red alga of the Haliptilon subulatum species or a salt thereof for its use in the modulation of the immune response in humans or animals. In a particular embodiment, a subject of the present invention is a sulphated polysaccharide extracted from a red alga of the Haliptilon subulatum species or a salt thereof for its use in the prevention and/or the treatment of inflammatory diseases in humans or animals.

The term “modulation of the immune response” has the meaning usually assigned thereto and well known to a person skilled in the art, in particular any property making it possible to stimulate or inhibit the immune reactions of the human or animal body.

The polysaccharides originating from the red algae are constructed on the basis of a linear chain of 3-β-galactopyranose and 4-α-galactopyranose units alternating regularly. The β-galactopyranose unit always belongs to the D series, whereas the α-galactopyranose unit has the D configuration in the carrageenans and the L configuration in the agarocolloids. Moreover, some of the 4-α-galactopyranose residues can exist in the form of 3,6-anhydrogalactose. The 3,6-anhydrogalactose form is obtained by elimination of the sulphate ester borne by carbon atom 6 of the α-galactose unit bound at position 4, under the action of galactose-6-sulphurylases during the biosynthesis or by an alkaline treatment.

In a particular embodiment of the invention, the sulphated polysaccharide is extracted from a red alga, advantageously from a red marine alga, advantageously a red marine alga of the class of the Florideophyceae, even more advantageously from a red marine alga of the Haliptilon subulatum species.

In a particular embodiment, the sulphated polysaccharide according to the invention, in which the sulphate content of said polysaccharide is less than or equal to 20% by weight of the polysaccharide, corresponds to formula (I):

[(unit A)-(unit B)]ₙ (I),

in which:
unit A is a 3-β-D-galactopyranose, in which the free hydroxyl functions are substituted with one or more groups selected from X_{A2}, X_{A4}, X_{A6},
unit B is selected from the group constituted by the B1 residue and the B2 residue:
   the B1 residue being a 4-α-D/L-galactopyranose, in which the free hydroxyl functions are substituted with one or more groups selected from, X_{B2}, X_{B3} and X_{B6} and,
   the B2 residue being a 4-α-3,6-anhydrogalactopyranose, in which the free hydroxyl functions of the 4-α-3,6-anhydrogalactopyranose are substituted with an X_{B2} group and,
   the B1 and B2 residues being distributed randomly in the polysaccharide and the B2 residue representing at the most 5% by weight of the polysaccharide,
unit A is bound to unit B by an O-glycosidic bond between the carbon atom at position 1 of unit A and the carbon atom at position 4 of unit B and,
unit B is bound to unit A by an O-glycosidic bond between the carbon atom at position 1 of unit B and the carbon atom at position 3 of unit A,
X_{A2}, X_{A4}, X_{A6}, X_{B2}, X_{B3} and X_{B6} are selected independently of each other and independently for each unit A and/or B in the group comprising:
   a hydrogen atom;
   a sulphate group;
   a pyruvate (—COO—CO—CH₃) group, said pyruvate group being bound to the X_{A4} group by its carbon atom at position 2 and to the X_{A6} group by its carbon atom at position 2;
   a saccharide unit bound to unit A or B by an O-glycosidic-type bond at position 1 (C₁) of the saccharide unit, the saccharide unit being selected from a galactose (or T-galactose), a xylose (or T-xylose), an arabinose (or T-arabinose) and a glucuronic acid (or T-glucuronic acid); and
   a (C₁-C₆) alkoxyl group, a (C₁-C₆) alkylcarbonyl group, a (C₁-C₆) alkoxycarbonyl group, a (C₁-C₆) acyloxy group, a group originating from a diacid, a phosphate group;
   a —CH₂Xa group, in which Xa represents a hydrogen atom, a hydroxy group, a (C₁-C₆) alkoxyl group, a (C₁-C₆) acyloxy group, a sulphate group;
n is an integer comprised between 10 and 3000.

Formulae (Ia) and (Ib) below illustrate the units (unit A)-(B1 residue) and (unit A)-(B2 residue) respectively:

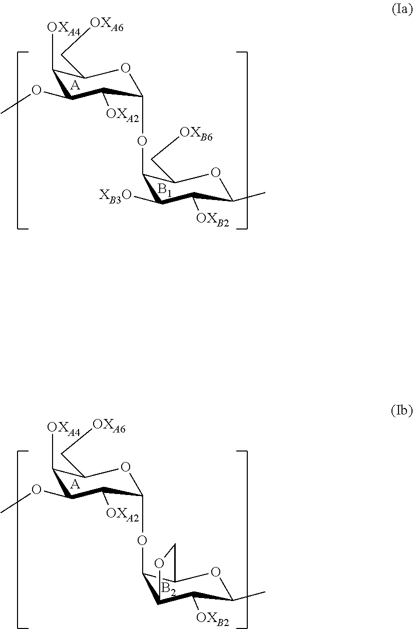

Within the meaning of the present invention, by “polysaccharide” is meant both a high-molecular-weight polysaccharide and a low-molecular-weight polysaccharide. By “high-molecular-weight polysaccharide” is meant a polysaccharide having a molecular weight comprised between 100 and 1,000 kDa. By “low-molecular-weight polysaccharide” is meant a polysaccharide having a molecular weight comprised between 5 and 100 kDa.

Within the meaning of the present invention, “n” represents an integer comprised between 10 and 3000, advantageously comprised between 10 and 2000, advantageously comprised between 10 and 1000, advantageously comprised between 10 and 900, advantageously comprised between 10 and 800, advantageously comprised between 10 and 700. Advantageously, “n” is comprised between 10 and 700, advantageously between 50 and 700, more advantageously between 70 and 650.

Within the meaning of the present invention, by “T-saccharide unit” is meant a saccharide unit bound to unit A or B by an O-glycosidic-type bond at position 1 (C₁) of the saccharide unit. Thus, by “T-galactose”, “T-xylose”, “T-arabinose” and “T-glucuronic acid” is meant respectively a galactose bound to unit A or B by an O-glycosidic-type bond at position 1 (C₁) of the galactose, a xylose bound to unit A or B by an O-glycosidic-type bond at position 1 (C₁) of the xylose, an arabinose bound to unit A or B by an O-glycosidic-type bond at position 1 (C₁) of the arabinose and a glucuronic acid bound to unit A or B by an O-glycosidic-type bond at position 1 (C₁) of the glucuronic acid.

Within the meaning of the present invention, by (C₁-C₆) alcoxyl or (C₁-C₆) alkoxyl or (C₁-C₆) alkyloxy group, the —OR— groups, R being a C₁-C₆ alkyl group, i.e. a straight or branched chain comprising from 1 to 6 carbon atoms. By way of examples of alkyls, the methyl, ethyl, propyl, butyl, isobutyl, tert-butyl, pentyl, isopentyl, tert-pentyl, hexyl and isohexyl groups may be mentioned. By way of examples of (C₁-C₆) alcoxyls, the methoxy (OCH₃), ethoxy, propoxy, isopropoxy, butoxy, isobutoxy, tert-butoxy, pentoxy, isopentoxy, tert-pentoxy, hexoxy and isohexoxy groups may be mentioned.

Within the meaning of the present invention, by (C₁-C₆) alkylcarbonyl group is meant the —COR groups, R being a C₁-C₆ alkyl group as defined previously. The methylcarbonyl, ethylcarbonyl, n-propylcarbonyl, iso-propylcarbonyl, n-butylcarbonyl, iso-butylcarbonyl, sec-butylcarbonyl, tert-butylcarbonyl, n-pentylcarbonyl, iso-pentylcarbonyl, neo-pentylcarbonyl, tert-pentylcarbonyl, n-hexylcarbonyl and iso-hexylcarbonyl groups may be mentioned by way of examples.

Within the meaning of the present invention, by (C₁-C₆) alkoxycarbonyl group is meant the COOR groups, R being a C₁-C₆ alkyl group as defined previously. The methoxycarbonyl (—COOCH₃), ethoxycarbonyl, propoxycarbonyl, isopropoxycarbonyl, butoxycarbonyl, isobutoxycarbonyl, tert-butoxycarbonyl, pentoxycarbonyl, isopentoxycarbonyl, tert-pentoxycarbonyl, hexoxycarbonyl and isohexoxycarbonyl groups may be mentioned by way of examples.

Within the meaning of the present invention, by (C₁-C₆) acyloxy group is meant the —OCOR groups, where R is a C₁-C₆ alkyl group as defined previously. The acetyloxy (—OCOCH₃) and propionyloxy groups may be mentioned by way of examples.

Within the meaning of the present invention, by group originating from a diacid is meant a group corresponding to the formula —OCOO—(CH₂)ₚ—COOH, where p is comprised between 0 and 4. Diacids from which these groups originate: the oxalate, malonate, succinate and glutarate groups, may be mentioned by way of examples.

Within the meaning of the invention, by “sulphate group” is meant a group of the (—SO₃H) type. Within the meaning of the invention, by “phosphate group” is meant a group of the (—PO₃H₂) type.

Within the meaning of the invention, by “sulphate group” is meant a group of the (—SO₃H) type or of the —SO₃⁻ type.

In an advantageous embodiment, the —X_{A2}, X_{A4}, X_{A6}, X_{B2}, X_{B3} and X_{B6} groups are selected independently of each other and independently for each unit A and/or B in the group comprising:
a hydrogen atom,
a sulphate group,
a pyruvate group, said pyruvate group being bound to the X_{A4} group by its carbon atom at position 2 and to the X_{A6} group by its carbon atom at position 2,
a saccharide unit bound to unit A or B by an O-glycosidic-type bond at position 1 (C₁) of the saccharide unit, and selected from a T-galactose, a T-xylose, a T-arabinose and a T-glucuronic acid.

In a more advantageous embodiment,
X_{A2}, X_{B2} and X_{B3} are selected from a hydrogen atom and a sulphate group;
X_{A4} is selected from a hydrogen atom, a sulphate group and a pyruvate group, said pyruvate group being bound to the X_{A4} group by its carbon atom at position 2 and to the X_{A6} group by its carbon atom at position 2;
X_{A6} is selected from a hydrogen atom, a sulphate group, a T-galactose saccharide unit bound to unit A by an O-glycosidic-type bond, a T-xylose saccharide unit bound to unit A by an O-glycosidic-type bond, a T-arabinose saccharide unit bound to unit A by an O-glycosidic-type bond and a T-glucuronic acid saccharide unit bound to unit A by an O-glycosidic-type bond, and
X_{B6} is selected from a hydrogen atom, a sulphate group, a T-galactose saccharide unit bound by an O-glycosidic-type bond to the B1 residue, a T-xylose saccharide unit bound by an O-glycosidic-type bond to the B1 residue, a T-arabinose saccharide unit bound by an O-glycosidic-type bond to the B1 residue and a T-glucuronic acid saccharide unit bound by an O-glycosidic-type bond to the B1 residue.

By “salt” is meant any addition salt with a mineral or organic acid by the action of such an acid within an organic or aqueous solvent such as an alcohol, a ketone or an ether, which does not cause allergic reactions when it comes into contact with the skin or any other part of the human or animal body. By way of examples of such salts, the following salts may be mentioned: benzenesulphonate, hydrobromide, hydrochloride, citrate, ethanesulphonate, fumarate, gluconate, iodate, isethionate, maleate, methanesulphonate, methylene-bis-b-oxynaphthoate, nitrate, oxalate, palmoate, phosphate, salicylate, sulphate, tartrate, theophyllinacetate and p-toluenesulphonate. In a particular embodiment, the salt is a cosmetically acceptable salt or a dermatologically acceptable salt.

In a particular embodiment of the invention, the sulphated polysaccharide can inhibit the release of VEGF by the cells involved in the inflammatory process.

In a particular embodiment of the invention, the sulphated polysaccharide can be a VEGF antagonist. By “VEGF antagonist” is meant a substance capable of reducing or completely inhibiting the release of VEGF. In a particular embodiment of the invention, the sulphated polysaccharide according to the invention is capable of reducing the release of VEGF by at least 15%, advantageously 20%, advantageously at least 25%, advantageously at least 30%, advantageously at least 35%. In a particularly advantageous embodiment of the invention, the sulphated polysaccharide according to the invention is capable of reducing the release of VEGF by 25% to 35%, advantageously 29% to 33%.

In a particular embodiment of the invention, the sulphated polysaccharide is capable of inhibiting or antagonizing the effects of VEGF in order to prevent and/or treat inflammatory diseases, in particular of the skin, while reducing the risks of side effects. Advantageously, the sulphated polysaccharide according to the invention or a salt thereof can be used in the prevention and/or treatment of inflammatory diseases in humans or animals.

In addition, as demonstrated in the examples, the sulphated polysaccharide according to the invention is capable of inhibiting the formation of the pseudotubes induced by the VEGF in a co-culture of human dermal endothelial cells (HMVECs) and of normal human dermal fibroblasts (NHDFs), of inhibiting the expression of the genes involved in angiogenesis in normal human epidermal keratinocytes (NHEKs) and in particular JAG1, VEGFA and CYR61. The sulphated polysaccharide is also capable of inhibiting cell proliferation in NHEK-type cells by stimulating the expression of the CDKN1C coding for an inhibitor of cell proliferation, as well as the expression of the CEBPA gene which codes for a CEBPA protein involved in regulation of the cell cycle. In addition, the sulphated polysaccharide induces in the same NHEK cells an inhibition of the expression of the genes coding for growth factors (HBEGF and VEGFA) or the genes involved in the regulation of cell proliferation (DUSP6, DUSP5, DUSP4, CDKN3).

In addition, these sulphated polysaccharides are obtained from algae, thus reducing the production costs and the risks of contamination.

In a particular embodiment of the invention, the sulphated polysaccharide has a molecular weight less than or equal to 500 kDa. Advantageously the sulphated polysaccharide according to the invention has a molecular weight less than or equal to 450 kDa, advantageously less than or equal to 400 kDa, advantageously less than or equal to 350 kDa, advantageously less than or equal to 300 kDa, advantageously less than or equal to 250 kDa, advantageously less than or equal to 200 kDa. Advantageously, the sulphated polysaccharide according to the invention has a molecular weight comprised between 10 kDa and 500 kDa, advantageously comprised between 10 kDa and 400 kDa, advantageously comprised between 10 kDa and 300 kDa, advantageously comprised between 10 kDa and 250 kDa.

In another particular embodiment of the invention, the sulphated polysaccharide has a sulphates content less than or equal to 20%. Advantageously the sulphates content of the sulphated polysaccharide is less than or equal to 19%, advantageously less than or equal to 18%, advantageously less than or equal to 17%, advantageously less than or equal to 16%. Advantageously the sulphates content of the sulphated polysaccharide is comprised between 8% and 20%, advantageously between 9% and 18%, advantageously between 10% and 16%, advantageously between 13% and 16%.

In another embodiment of the invention, the sulphated polysaccharide has a 3,6-anhydrogalactopyranose residues content less than or equal to 5%. In an advantageous embodiment, the sulphated polysaccharide has a 3,6-anhydrogalactopyranose residues content less than or equal to 4%, advantageously less than or equal to 3%, advantageously less than or equal to 2%, advantageously less than or equal to 1.5%. Even more advantageously, the sulphated polysaccharide has a 3,6-anhydrogalactopyranose residues content less than or equal to 1.4%.

The present invention also relates to compositions, in particular cosmetic and/or dermatological compositions making it possible to reduce vascular anomalies by means of the combination of active agents capable of inhibiting the formation of pseudotubes induced by the VEGF and reducing the destruction of the fibres of the matrix.

In a particular embodiment, the composition can be a cosmetic composition or a dermatological composition.

By “cosmetic composition” is meant any substance or preparation intended to be applied to the skin, in order to clean it, protect it, keep it in good condition, modify its appearance, perfume it or correct its odour.

By “dermatological composition” is meant any substance or preparation intended to be applied to the skin, the mucous membranes and the epithelial appendages (nails, hair, fur) in order to prevent the occurrence of skin disorders and/or to treat them.

In the compositions according to the invention, the sulphated polysaccharide is used in a quantity ranging from 0.000001% to 10% by weight with respect to the total weight of the composition, advantageously in a quantity ranging from 0.0001% to 5% by weight with respect to the total weight of the composition. Even more advantageously, the sulphated polysaccharide is used in a quantity ranging from 1% to 5% by weight with respect to the total weight of the composition.

In a known manner, the composition of the invention can also contain at least one pharmaceutically, dermatologically or cosmetically acceptable excipient that is usual in the fields considered, such as the hydrophilic or lipophilic gelling agents, hydrophilic or lipophilic compounds, oils, emulsifiers, preservatives, antioxidants, solvents, perfumes, fillers, filters, odour absorbers and colorants. The quantities of these different excipients are those conventionally used in the fields considered, and for example from 0.01% to 10% of the total weight of the composition. These excipients, depending on their nature, can be introduced into the fatty phase, into the aqueous phase and/or into the lipid spherical particles.

As oils that can be used in the invention, the mineral oils (vaseline oil), vegetable oils (liquid fraction of shea butter, sunflower oil), animal oils (perhydrosqualene), synthetic oils (Purcellin oil), silicone oils (cyclomethicone) and fluorinated oils (perfluoropolyethers) may be mentioned. Fatty alcohols and fatty acids (stearic acid) may be added to these oils. As emulsifiers that can be used in the invention, glycerol stearate, polysorbate 60 and the PEG-6/PEG-32/glycol stearate mixture may be mentioned for example. As solvents that can be used in the invention, the lower alcohols, in particular ethanol and isopropanol, may be mentioned. As hydrophilic gelling agents, the carboxyvinyl polymers (carbomers), and the acrylic copolymers such as the copolymers of acrylates/alkylacrylates, the polyacrylamides, the polysaccharides such as hydroxypropylcellulose, the natural gums and clays may be mentioned and, as lipophilic gelling agents, the modified clays such as the bentones, metal salts of fatty acids such as aluminium stearates, hydrophobic silica, polyethylenes and ethyl cellulose may be mentioned. As hydrophilic compounds, the proteins or protein hydrolysates, the amino acids, polyols, urea, allantoin, sugars and sugar derivatives, vitamins, starch, plant extracts, in particular aloe vera, and hydroxyacids can be used.

As lipophilic compounds, tocopherol (vitamin E) and derivatives thereof, retinol (vitamin A) and derivatives thereof, the essential fatty acids, ceramides and essential oils may be used.

It is also possible to combine the sulphated polysaccharides with active agents, in particular, anti-redness agents, decongestants, antibacterial agents, antiseptics and antimicrobials, anti-inflammatories, anti-irritant and/or soothing agents, cicatrizants and/or restructurants of the skin barrier, antioxidants, moisturizing/emollient agents, anti-aging agents, mineral or organic sun filters and screens, sun protection active agents.

Among these active agents, there may be mentioned by way of examples:
the anti-redness agents such as the lupin peptides, permethol, genistein, esculoside, dextran sulphate, hesperidin methyl chalcone, the retinoids, licochalcone, oxymetazoline, kinetin, liquorice extract, vitamin P-like substances, butcher's broom extract, Sophora japonica, witch hazel extract, ruscus, the antibiotics such as doxycyclin, the polyphenols including tannins, phenolic acids, anthocyans, procyanidols, flavonoids with, for example, quercetin, extracts of green tea, red fruits, cocoa, grape, Passiflora incarnata, and Citrus;
the antiseptics such as salicylic acid and derivatives thereof (n-octanoyl-5 salicylic acid), or crotamiton;
the antibacterial agents such as clindamycin phosphate, erythromycin or antibiotics of the class of the tetracyclines;
the antiparasitic agents, in particular metronidazole or the pyrethrinoids;
the antifungal agents, in particular the compounds belonging to the class of the imidazoles such as econazole, ketoconazole or miconazole or salts thereof, the polyene compounds such as amphotericin B, the compounds of the family of the allylamines, such as terbinafine, or also octopirox;
the steroidal anti-inflammatory drugs (SAIDs), such as the corticoids, hydrocortisone, betamethasone valerate or clobetasol propionate, or the antisense agents: salts, acetylsalicylic acid, acetaminophen or glycyrrhetinic acid or the non-steroidal anti-inflammatory drugs (NSAIDs);
the anaesthetic agents such as lidocaine hydrochloride and derivatives thereof;
the antipruritic agents such as thenaldine, trimeprazine or cyproheptadine;
the anti-free radical agents, such as alpha-tocopherol or esters thereof, the superoxide dismutases, certain metal chelating agents, or ascorbic acid and esters thereof;
the keratolytic agents such as 13-cis or all-trans retinoic acid, benzoyl peroxide or the hydroxyacids;
the antiviral agents such as acyclovir and valacyclovir;
the anti-irritant and/or soothing agents such as glycyrrhetinic acid (liquorice derivatives) with salts and esters thereof, lipoic acid, beta-carotene, vitamin B3 (niacinamide, nicotinamide), vitamin E, vitamin C, vitamin B12, lycopene or lutein, spring or thermal waters (Avène water, Roche Posay water, Saint Gervais water, Uriage water, Gamarde water), or also topical disulone. The isoflavones, in particular of soya, such as for example genistein/genistin, daidzein/daidzin may also be mentioned;
the cicatrizants and/or restructurants of the skin barrier such as panthenol (vitamin B5) zinc oxide, madecassic or asiatic acid, dextran sulphate, co-enzyme Q10, glucosamine and derivatives thereof, chondroitin sulphate and the glycosaminoglycanes overall, dextran sulphate, the ceramides, cholesterol, squalane and phospholipids. Agonists of the peroxisome proliferator-activated receptors (PPARs) such as rosiglitazone or pioglitazone, retinoid X receptor (RXR) agonists, and oxysterol receptor (LXR) agonists can also be used;
the antioxidants such as the thiols and the phenols, the liquorice derivatives such as glycyrrhetinic acid with salts and esters thereof, alpha bisabolol, lipoic acid, vitamin B12, vitamin B3 (niacinamide, nicotinamide), the C vitamins, the E vitamins, co-enzyme Q10, krill, glutathion, BHT (butylated hydroxytoluene), BHA (butylated hydroxyanisole), lycopene or lutein, and beta-carotene. In the group of the antioxidants, anti-glycation substances are also found, such as carnosine or certain peptides, n-acetylcysteine, as well as the antioxidant or antiradical enzymes such as SOD (superoxide dismutase), catalase, glutathione peroxidase, thioredoxin reductase and agonists thereof;
the moisturizing/emollient agents such as glycerin or derivatives thereof, urea, pyrrolidone carboxylic acid and derivatives thereof, hyalouronic acid of any molecular weight, the glycosaminoglycans, and any other polysaccharides of marine, vegetable or biotechnological origin, such as for example xanthan gum, fucogel®, fatty acids such as lauric acid, mystiric acid, the omega 3,6 and 7,9-type poly- and mono-unsaturated fatty acids, such as linoleic acid and palmitoleic acid, certain butters such as shea butter;
the anti-aging agents such as the C vitamins, hyaluronic acid of any molecular weight, the retinoids such as retinol, retinal and the retinoids; in particular the non-aromatic retinoids such as retinaldehyde, tretinoin, isotretinoin and 9-cis retinoic acid, vitamin A, the monoaromatic retinoids such as etretinate, all-trans acitretin and motrerinide, and the polyaromatic retinoids such as adapalene, tazarotene, tamibarotene and arotinoid methyl sulphone;
the sun protection active agents, in particular the UVB and/or UVA sun filters or screens; such as the mineral and/or organic screens or filters known to a person skilled in the art who will adapt their selection and their concentrations depending on the degree of protection sought. By way of examples of sun protection active agents, titanium dioxide, zinc oxide, methylene bis-benzotriazolyl tetramethylbutylphenol (trade name: TINOSORB M®) and bis-ethylhexyloxyphenol methoxyphenyl triazine (trade name: TINOSORB S®), octocrylene, butyl methoxydibenzoylmethane, terephthalylidene dicamphor sulphonic acid, 4-methylbenzylidene camphor, benzophenone, ethylhexyl methoxycinnamate, ethylhexyl dimethyl PABA, diethylhexyl butamido triazone may in particular be mentioned.

The compositions according to the invention can be presented in all the forms known to a person skilled in the art and adapted to the route of administration, in particular by injection, by oral route, by topical route, or also in the form of supplements and/or food products. Advantageously, the compositions according to the invention can be administrable by oral, topical or injectable route or in the form of supplements and/or food products.

For a topical application, the composition can be presented in particular in the form of aqueous, hydroalcoholic or oily solutions, or of lotion- or serum-type dispersions, of milk-type emulsions with a liquid or semi-liquid consistency, obtained by dispersing a fatty phase in an aqueous phase (Oil in Water) or vice-versa (Water in Oil), or of suspensions or cream- or gel-type emulsions with a soft, semi-solid or solid consistency, of microemulsions, or also of microcapsules, of microparticles, or of ionic- and/or non-ionic type vesicular dispersions. They can also be packaged in the form of aerosol or spray compositions also containing a propellant under pressure. These compositions are prepared according to the usual methods. They can also be used for the scalp in the form of aqueous, alcoholic or hydroalcoholic solutions, or in the form of creams, gels, emulsions, foams or also of aerosol compositions. The injectable compositions can be presented in the form of an aqueous or oily lotion, or in the form of serum. For ingestion by oral route, numerous embodiments and in particular food supplements are possible. Their formulation is achieved by the usual methods for producing tablets, gelatin capsules, capsules, coated tablets, emulsions, gels and syrups. In particular the sulphated polysaccharide and the other active ingredients of the invention can be incorporated into all forms of food supplements or of enriched foods, for example, food bars, compacted or non-compacted powders, drinks, dairy products and in particular yogurts and drinking yogurts. The powders can be diluted in water, sodas, fruit juices, dairy or soya- or rice-based products, or be incorporated into food bars.

The operating conditions for preparing these compositions according to the invention form part of the general knowledge of a person skilled in the art.

The quantities of the different constituents of the compositions according to the invention are those conventionally used in the fields considered. These compositions constitute, in particular, creams for protection, treatment or care for the face, for the hands, for the feet, for the large anatomical folds, or for the body, body lotions for protection or care, lotions, gels or foams for care of the skin and mucous membranes, such as cleansing or disinfecting lotions, compositions for the bath, and compositions containing a bactericidal agent. The compositions can also consist of solid preparations constituting soaps or cleansing bars.

When the composition of the invention is an emulsion, the proportion of the fatty phase can range from 5% to 80% by weight, and advantageously from 5% to 50% by weight with respect to the total weight of the composition. The oils, the emulsifiers and the co-emulsifiers used in the composition in the form of emulsion are selected from those conventionally used in the dermatological field. The emulsifier and the co-emulsifier are present, in the composition, in a proportion ranging from 0.3% to 30% by weight, and advantageously from 0.5% to 20% by weight with respect to the total weight of the composition. The emulsion can, moreover, contain lipid vesicles.

When the composition is a solution or an oily gel, the quantity of oil can range up to more than 90% by weight with respect to the total weight of the composition.

Another aspect of the invention relates to a dermatological composition comprising at least one sulphated polysaccharide extracted from a red alga of the Haliptilon subulatum species or a dermatologically acceptable salt thereof, as described previously, for its use in the prevention, reduction and treatment of inflammatory diseases, in particular of the skin.

The words “treat” or “treatment” or “curative treatment” are defined by a treatment leading to a cure or a treatment alleviating, improving and/or eliminating, reducing and/or stabilizing the symptoms of a disease or the suffering that it causes.

By “inflammatory disease of the skin” or “dermatitis” is meant any disease leading to an inflammation of the skin, characterized by the presence on the skin of redness, swelling, heat or pain. These diseases can be due to an infection, for example by a microbe, a parasite, a virus or a fungus, to an inflammation of the joints, to allergies, to mechanical or chemical attacks, such as ultraviolet-type radiation, or X-ray-type radiation. By way of examples, there may be mentioned as inflammatory diseases of the skin: psoriasis, atopical dermatitis, rosacea, couperose, acne, vulgar warts, bullous skin diseases, contact eczema, skin cancers, redness, erythema, telangiectasia, inflammation of the skin associated with UV exposure, such as photoirritation, photosensitization, photoaging, photocarcinogenesis, venous lymphatic insufficiency or heavy legs syndrome, this list not being limitative.

Another aspect of the invention relates to a method for preventing and/or treating inflammatory diseases of the skin in a patient, comprising the administration to said patient of a dermatological composition comprising at least one sulphated polysaccharide extracted from a red alga of the Haliptilon subulatum species or a cosmetically acceptable salt thereof, as described previously.

Another aspect of the invention relates to a method for preventing and/or treating inflammatory diseases, in particular of the skin, comprising the administration to a patient requiring it, of a dermatological composition comprising at least one sulphated polysaccharide extracted from a red alga of the Haliptilon subulatum species or a dermatologically acceptable salt thereof, as described previously.

Another aspect of the invention relates to a cosmetic composition comprising at least one sulphated polysaccharide extracted from a red alga of the Haliptilon subulatum species or a cosmetically acceptable salt thereof, as described previously, for its use in the prevention, reduction and treatment of the appearance of redness on the skin.

Due to its anti-angiogenic properties, the sulphated polysaccharide also makes it possible to reduce the reactivity of certain types of skin likely to develop redness, and thus to prevent and treat the appearance of this redness. The sulphated polysaccharide is extracted from a red alga of the Haliptilon subulatum species; one of the advantages of the invention is therefore to offer to persons subject to skin redness and thus having sensitive skin, a composition comprising substantially products of natural origin.

By “redness of the skin” is meant erythema, in particular facial erythema and telangiectasias, of all origins.

By “prevent redness of the skin” or “prevention of redness of the skin” or “prophylaxis of redness of the skin” or “preventative treatment of redness of the skin” or “prophylactic treatment of redness of the skin” is meant both a treatment leading to the prevention of unsightly redness of the skin and a treatment reducing and/or delaying the incidence of redness of the skin or the risk of it occurring. By “prevent redness of the skin” or “prevention of redness of the skin” or “prophylaxis of redness of the skin” or “preventative treatment of redness of the skin” or “prophylactic treatment of redness of the skin” is also meant any action making it possible to avoid or at the very least to reduce the formation of unsightly redness by application of the composition before and during the course of an event known to be able to cause the appearance of redness, such as sun exposure or stress.

By “person likely to develop redness or having redness” is meant a person having redness, irrespective of the location thereof on the body and in particular on the face, and irrespective of the stage at which this redness can be classified from a clinical point of view. This redness can be deemed unsightly or debilitating for this person.

Another aspect of the invention relates to a method for preventing and/or treating the appearance of redness on the skin, comprising the administration to a patient requiring it, of a dermatological composition comprising at least one sulphated polysaccharide extracted from a red alga of the Haliptilon subulatum species or a dermatologically acceptable salt thereof, as described previously.

Another aspect of the invention relates to a method of cosmetic care comprising the application to the skin of the cosmetic composition as described previously, for the prevention, and/or treatment of the appearance of redness on the skin.

By “application” is meant any act making it possible to cause the composition according to the invention to be absorbed on the patient, by any route, form or method of administration.

Another aspect of the present invention relates to a process for obtaining sulphated polysaccharide. The sulphated polysaccharide according to the invention can be obtained by processes well known to a person skilled in the art. In particular, the extraction of a high-molecular-weight sulphated polysaccharide can in particular be carried out by a process comprising the following steps:
a) Dispersion in water of a powder of red alga, in particular a powder of previously dried Haliptilon subulatum;
b) Precipitation by at least one polar solvent of the high-molecular-weight polysaccharides;
c) Drying of the precipitate containing the high-molecular-weight polysaccharides.

In a particular embodiment, step b) of alcohol precipitation is carried out using ethanol or isopropanol. In a particular embodiment, the step of drying the precipitate (step c) can be carried out by lyophilization or using an oven, in particular at a temperature comprised between 40° C. and 75° C., advantageously 50° C. overnight. The method of obtaining according to the invention can be repeated several times in order to obtain a satisfactory degree of purity of the polysaccharide.

The extraction process according to the present invention makes it possible to obtain polysaccharides in the form of a fine creamy white powder with a production yield of the order of 10-20% with respect to the dry weight of the powder of the alga Haliptilum subulatum used.

In another particular embodiment, the low-molecular-weight sulphated polysaccharides are prepared by acid degradation of high-molecular-weight polysaccharides. The low-molecular-weight sulphated polysaccharides can also be obtained by depolymerization techniques well known to a person skilled in the art such as radical or enzymatic depolymerizations.

In a particular embodiment, the process for extracting a low-molecular-weight sulphated polysaccharide from the alga Haliptilon subulatum comprises the following steps:
a) Dispersion of the powder of high-molecular-weight polysaccharides in an aqueous solution with an acid pH, in particular a pH comprised between 3 and 6.5;
b) Precipitation by at least one polar solvent of the low-molecular-weight polysaccharides;
c) Drying of the precipitate containing the low-molecular-weight polysaccharides.

is In a particular embodiment, the aqueous solution used in the dispersion step (step a)) is a hydrochloric acid (HCl) solution. In a particular embodiment, the HCl solution has a concentration comprised between 1M and 5M, advantageously 2M, at a temperature ranging from 50° C. to 100° C. and under stirring for 30 to 60 minutes. In a particular embodiment, step b) of alcohol precipitation is carried out using ethanol or isopropanol. In a particular embodiment, the step of drying the precipitate (step c)) is carried out by lyophilization or using an oven, in particular at a temperature comprised between 40° C. and 75° C., advantageously 50° C. overnight.

The sulphated polysaccharides extracted from red algae according to the invention have the advantage of having no contamination and safety problems. In addition, these sulphated polysaccharides offer an economic advantage. The approximate yield of sulphated polysaccharides of the present invention is approximately 10% to 20% with respect to the initial dry weight of algae from which they have been extracted. Moreover, the red algae, in particular of the class of the Florideophyceae, in particular the Haliptilon subulatum species, are easy to cultivate, which also contributes to the low cost price of the final product.

The following figures and examples illustrate the invention without, however, limiting it.

### FIGURES

FIG. 1: Effects of the sulphated polysaccharide on the formation of pseudotubes in the basal condition or stimulated with VEGF at 10 ng/ml according to Example 6.

FIG. 2: Effects of the sulphated polysaccharide on the viability of normal human keratinocytes according to Example 8.

FIG. 3: Effects of the sulphated polysaccharide on the release of VEGF by normal human keratinocytes, relative to the total quantity of proteins according to Example 9.

FIG. 4: Effects of the sulphated polysaccharide on the release of IL8 by normal human keratinocytes, relative to the total quantity of proteins according to Example 9.

### EXAMPLES

### Example 1

### Extraction of the High-Molecular-Weight Polysaccharides (HMWP)

By “high-molecular-weight polysaccharide” or “HMWP” is meant a polysaccharide having a molecular weight comprised between 100 and 1,000 kDa.

The extraction of the high-molecular-weight polysaccharides is carried out by dispersing 100 grams of powder of the alga Haliptilon subulatum in 1 litre of water at 90° C. under vigorous stirring (500 rpm) for 4 hours. The mixture is then filtered hot using diatomaceous earth (100 g) on a frit glass (porosity 1, more precisely 100 to 160 μm). The filtrate is then centrifuged (10,000 g, 30 minutes) at ambient temperature in order to obtain the alga extract enriched with polysaccharides. The Haliptilon subulatum extract is then precipitated from 3 volumes of ethanol 96% (at 4° C.) under stirring (500 rpm) for 2 hours.

The precipitate is recovered by filtration on frit glass (porosity 1 or 2, more precisely 100 to 160 μm or 40 to 100 μm respectively) or centrifugation (10,000 g, 30 minutes) at ambient temperature then washed with acetone (50 to 100 mL). The precipitate is then recovered by filtration on frit glass (porosity 2, more precisely 40 to 100 μm) or centrifugation (10,000 g, 30 minutes) at ambient temperature then dried in an oven at 50° C. overnight. Finally, the precipitate is ground (in a blender) in order to obtain a fine powder of high-molecular-weight polysaccharides extracted from Haliptilon subulatum.

The yield of high-molecular-weight polysaccharides thus obtained is of the order of 10 to 20% with respect to the dry weight of powder of the alga Haliptilon subulatum used.

### Example 2

### Extraction of the Low-Molecular-Weight Polysaccharides (LMWP)

By “low-molecular-weight polysaccharide” or “LMWP” is meant a polysaccharide having a molecular weight comprised between 5 and 100 kDa.

The production of the low-molecular-weight polysaccharides is carried out by dispersing 2.5 grams of powder of high-molecular-weight polysaccharide (extracted from Haliptilon subulatum) in 125 mL of HCl (2M) at 100° C. under vigorous stirring (500 rpm) for 1 hour. The mixture is then cooled to ambient temperature then neutralized with soda (5 M). The medium is precipitated from 7 volumes of ethanol 96% (at 4° C.) under stirring (500 rpm) for 2 hours. The precipitate is recovered by filtration on frit glass (porosity 1 or 2, more precisely 100 to 160 μm or 40 to 100 μm respectively) or centrifugation (10,000 g, 30 minutes) at ambient temperature then washed with acetone (50 mL). The precipitate is then recovered by filtration on frit glass (porosity 2, more precisely 40 to 100 μm) or centrifugation (10,000 g, 30 minutes) at ambient temperature then dried in an oven at 50° C. overnight. Finally, the precipitate is ground (in a blender) in order to obtain a fine powder of low-molecular-weight polysaccharides extracted from Haliptilon subulatum.

The yield of low-molecular-weight polysaccharides thus obtained is of the order of 70% with respect to the dry weight of powder of high-molecular-weight polysaccharides and 14% with respect to the dry weight of powder of the alga Haliptilon subulatum used.

### Example 3

### Determination of the Molecular Weights of the High- (HMWP) and Low- (LMWP) Molecular-Weight Polysaccharides

The high- and low-molecular-weight polysaccharides are prepared according to the conditions described previously (Examples 1 and 2). The molecular weights of the sulphated polysaccharides are determined according to the following protocol:
a) Solubilization of the polysaccharides powder at a level of 0.5 to 10 g/L in an aqueous solution of ultrapure quality at a temperature ranging from 4° C. to 60° C. and under stirring for 30 minutes to 48 hours;
b) Filtration of the samples on membrane with a porosity of 0.45 μm;
c) Injection and analysis by size-exclusion chromatography coupled with light scattering (SEC/MALLS);
d) The technique makes it possible to access number average molecular weights (Mn) and weight average molecular weights (Mw) and also provides information on the shape and dimension of the chains and the polydispersity (PI).

TABLE 1
Summary of the weight and number average molecular
weights observed for HMWP and LMWP.

| | Mn | Mw | PI |
|---|---|---|---|
| | (g/mol) | (g/mol) | (Mw/Mn) |
| HMWP | 133300 | 213500 | 1.6 |
| LMWP | 12840 | 36640 | 2.8 |

This example shows that the molecular weight of the high-molecular-weight polysaccharides is of the order of 214 kDa and that the molecular weight of the low-molecular-weight polysaccharides is of the order of 37 kDa.

### Example 4

### Determination of the Sulphates and 3,6-anhydrocialactopyranose Residues Content of the Polysaccharides of the Invention

### 1. Determination of the Sulphates Content

### Assay by Turbidimetry (BaCl₂/Gelatin)

The sulphate ions released during the hydrolysis of the polysaccharides will form, in the presence of barium chloride (BaCl₂, 2H₂O) and of gelatin, a precipitate of barium sulphate, the appearance of which is measured at 550 nm, as described in the publication by Dodgson & Price (Dodgson & Price, 1962, Biochemical Journal 84: 106-110).

150 mg of gelatin is dissolved in 50 mL of milli-Q water at 70° C. After cooling for 16 hours at 4° C., 0.5 g of BaCl₂ is added to the gelatin solution. 120 mg of lyophilized polysaccharide is hydrolyzed using 3 mL of 2 M HCl for 2 hours at 100° C. The mixture is centrifuged at 13,000 g for 30 minutes. 1 mL of supernatant is mixed with 9 mL of milli-Q water, 1 mL of 0.5 M HCl and 0.5 mL of BaCl₂/gelatin reagent. After 30 minutes at ambient temperature, the mixture is stirred and the absorbance is read immediately at 550 nm. The standard range is produced using a stock solution of K₂SO₄ at 3 mg/mL.

### Assay Using Azure A

The quantity of sulphates was determined using the colorimetric assay method developed by Jaques et al. (Jaques L. B. et al., 1968, Canadian Journal of Physiology and Pharmacology 46, pages: 351-360). In the aqueous phase, 3-amino-7-(dimethylamino)phenothizin-5-ium chloride (Azure A) complexes the sulphates that may be present, in particular within the polysaccharides composing the SPE fractions. The medium then develops a pink-violet colour absorbing at λ=535 nm, due to the formation of a chromophore in the presence of sulphates. The assay is semi-quantitative and gives an order of magnitude (˜mg) of the concentration of sulphates in a sample. 200 μL of solution to be assayed is introduced into plastic spectrophotometer cuvettes. 2 mL of aqueous solution of Azure A at 10 mg/L is added, then the sample is stirred. The absorbance is measured at λ=535 nm.

The quantification of the sulphates is determined from the calibration range of dextran sulphate (17% sulphated) and correction of the degree of sulphation of the latter (17 mg of sulphates per 100 mg of dextran sulphate).

TABLE 2
Summary of the sulphates content for
HMWP and LMWP.

| | Sulphates | |
|---|---|---|
| | content | Standard |
| | (% m/m) | deviation |
| HMWP | 13.5 | 0.09 |
| LMWP | 15.7 | 0.26 |

This example shows that the sulphates content of the high-molecular-weight polysaccharides is of the order of 13.5% and that the sulphates content of the low-molecular-weight polysaccharides is of the order of 15.7%.

### 2. Determination of the 3,6-anhydrodalactopyranose Residues Content

The most reproducible colorimetric method for assaying the 3,6-anhydrogalactose residues is that which uses a reagent based on resorcinol (see Yaphe & Arsenaut, 1965, Analytical Biochemistry 13, pages 143-148). The pink colouring which develops during the reaction is monitored at 555 nm. Three solutions are necessary for carrying out this assay: (i) a solution of acetaldehyde prepared by diluting 1 mL of acetaldehyde in 100 mL of ultrapure water (stable for approximately 1 month); (ii) a solution of resorcinol prepared by dissolving 150 mg of resorcinol in 100 mL of ultrapure water (stable for 7 days, away from light) and (iii) a solution of 10 M HCl.

For the assay, 50 to 100 μL of the polysaccharide solution to be assayed) is introduced into glass tubes. The volume is topped up to 200 μL using milli-Q water. The resorcinol reagent is prepared extemporaneously by adding 9 mL of the resorcinol solution and 1 mL of the acetaldehyde solution diluted to 1/25, to 100 mL of 10 M HCl. This reagent is only stable for 3 hours away from light. 1 mL of the resorcinol reagent is added to 200 μL of the polysaccharide solution to be assayed. After stirring, the tubes are left to rest for 4 minutes, then placed in a water bath at 80° C. for 10 minutes. They are then transferred to an ice bath for 1 minute 30 seconds. The absorbance must be read within the following 15 minutes at 555 nm.

D-fructose (solutions from 10 to 70 μg/mL) is used as standard. In fact it has been demonstrated that the absorbance curves at 555 nm depending on the monosaccharide concentration of the D-fructose and of the 3,6-anhydrogalactose are identical (see Yaphe & Arsenaut, 1965, Analytical Biochemistry 13, pages 143-148).

TABLE 3
Summary of the 3,6-anhydrodalactopyranose (anhydroG)
residues content for HMWP.

| | Content of (3,6) | |
|---|---|---|
| | anhydroG | Standard |
| | (% m/m) | deviation |
| HMWP | 1.32 | 0.013 |

This example shows that the 3,6-anhydrogalactopyranose content of the high-molecular-weight polysaccharides is 1.32%.

### Example 5

### Determination of the Composition in Terms of Monosaccharides and of the Structure of the Polysaccharides According to the Invention

10 mg of polysaccharides is dissolved in 1 mL of 2 M trifluoracetic acid for 90 minutes at 120° C., with manual stirring every 30 minutes. The samples are evaporated under a stream of nitrogen in order to remove excess traces of acid. 1 mL of methanol is added, then the sample is vortexed and evaporated under a stream of nitrogen. This step is repeated twice in order to remove residual traces of acids. Derivatization is carried out using BSTFA:TMCS (99:1). 400 μL of pyridine and 400 μL of N,O-bis(trimethylsilyl) trifluoroacetamide:trimethylchlosylane (BSTFA:TMCS) (99:1) are added per 2 mg of monosaccharides. The samples are then mixed, then placed at ambient temperature for 2 hours under stirring (450 rpm).

The samples are evaporated under a stream of nitrogen, then the trimethylsilyl-O-glycoside residues are taken up in 500 μL of dichloromethane. In this step, it is possible to dilute the sample to a greater or lesser extent. The standards (L-Rha, L-Fuc, L-Ara, D-Xyl, D-Man, D-Gal, D-Glc, D-GlcA, D-GalA) are prepared under the same conditions, in at least three different concentrations.

The trimethylsilylated derivatives are analyzed by gas chromatography coupled with mass spectrometry on an OPTIMA-1 MS column (30 m, 0.32 mm, 0.25 μm) with a helium flow rate of 2.3 mL/minute (up to 3 mL/min). The helium pressure is fixed at 8.8 psi, i.e. 60673.9 Pa, and the injection ratio at 25:1 (or 50:1). The temperature rise is 8° C./minute up to 100° C., over 3 minutes. Another temperature rise of 8° C./minute up to 200° C., maintained for 1 minute is programmed. The procedure concludes with a temperature rise of 5° C./minute up to 250° C. The ionization is carried out by Electron Impact (EI, 70 eV), the temperature of the trap is fixed at 150° C. and the targeted ions between 40 and 800 m/z.

TABLE 4
Composition of the HMWP sample in terms of monosaccharides.
Monosaccharides (mol %)*

| Gal | Ara | Xyl | GlcA | Glc |
|---|---|---|---|---|
| 94.1 | 1.51 | 1.88 | 1.51 | 1.0 |

*Composition in terms of monosaccharides estimated by GC/MS-IE. Gal: Galactose; Ara: Arabinose, Xyl: Xylose, GlcA: Glucoronic acid, Glc: Glucose.

### Example 6

### Effect of the High-Molecular-Weight Sulphated Polysaccharide According to the Invention on the Formation of Pseudotubes in an HMVEC+NHDF Co-Culture

The effects of the high-molecular-weight sulphated polysaccharide (HMWP) on the formation of pseudotubes was studied in a co-culture of human dermal endothelial cells (HMVECs) and normal human dermal fibroblasts (NHDFs), in basal condition or stimulated by VEGF (analysis by immunolabelling in situ).

### Culture and Treatment

The HMVEC and NHDF cells in co-culture were seeded in 96-well plates and cultured for 24 hours in culture medium.

The culture medium used is as follows:
EBM-2 (Endothelial cell basal medium 2) supplemented with 5% foetal calf serum (FCS), rhEGF, rhFGF, R3 IGF-1, hydrocortisone, vitamin C, gentamycin,
DMEM supplemented with 2 mM L-glutamine, 50 U/ml penicillin, 50 μg/ml streptomycin and 10% FCS.

The medium was then replaced with test medium containing or not containing (control) the compound to be tested and/or the VEGF inductor reference tested at 100 ng/ml; the compound was tested simultaneously in basal and stimulated condition (in the absence and in the presence of VEGF).

The test medium used was as follows:
EBM-2 (Endothelial cell basal medium 2) supplemented with rhEGF, rhFGF, R3 IGF-1, hydrocortisone, vitamin C, gentamycin,
DMEM supplemented with 2 mM L-glutamine, 50 U/ml penicillin, 50 μg/ml streptomycin and 1% FCS.

The cells were then incubated for 7 days, repeating the treatment after incubating for 72 hours. All the experimental conditions were produced in triplicate (n=3).

### In Situ Immunolabelling of the Pseudotubes

After incubation, the culture medium was removed and the cells were rinsed, fixed and permeabilized. The cells were then labelled with anti-VWF (Von Willebrand Factor) primary antibody. This antibody was revealed by a secondary antibody coupled with a fluorochrome (GAR-Alexa 488). Simultaneously, the cell nuclei were coloured with Hoechst 33258 (bis-benzimide).

The formation of the pseudotubes was observed using a NIKON Diaphot 300 microscope (×4 objective lens). The digital images (1 photo per well) were then recorded with a NIKON DS-Ri1 camera and the NIS-Elements 4.13.04 software.

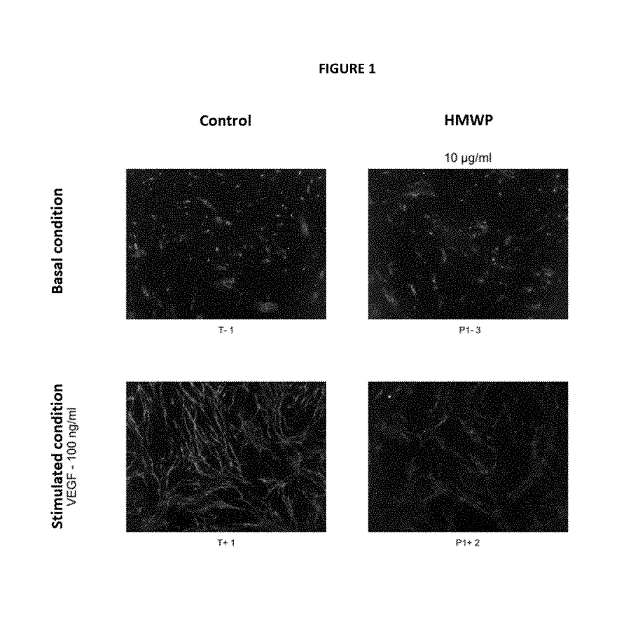

The labelling was quantified by measuring the entire surface of the pseudotubes using ImageJ software. The results of the labelling are presented in FIG. 1.

### Results

The formation of pseudotubes in the co-culture of endothelial cells (HMVECs) and dermal fibroblasts (NHDFs) after incubating for 7 days was measured by image analysis following immunolabelling with an anti-vWF antibody, the vWF being specifically expressed by the HMVEC cells.

The stimulation percentage is calculated according to the following formula:

$$Stimulation ( \% ) = ( \frac{Value}{Average of the control} \times 100 ) - 100$$

The inhibition percentage is calculated according to the following formula:

$$Inhibition ( \% ) = \frac{Average stimulated control - Value}{Average stimulated control - Average basal control} \times 100$$

TABLE 5
Effect of the HMWP sulphated polysaccharide according to the invention
on the formation of the pseudotubes—Basal condition.

| | | | | Basic data | | | | | | | | |
|---|---|---|---|---|---|---|---|---|---|---|---|---|
| | | | | Surface | | | | | | | | |
| Treatment | | | | of the | Average | | | | | Standardized data | | |
| Compounds | | | | pseudotubes | Surface | sem | % | sem | | % | sem | |
| tested | Concentration | | | (mm²) | (mm²) | (mm²) | Control | (%) | p⁽¹⁾ | Stimulation | (%) | p⁽¹⁾ |
| Control | — | | T − 1 | 0.16 | 0.12 | 0.02 | 100 | 16 | — | 0 | 16 | — |
| medium | | | T − 2 | 0.09 | | | | | | | | |
| without | | | T − 3 | 0.12 | | | | | | | | |
| VEGF | | | | | | | | | | | | |
| Medium | 100 | ng/ml | T + 1 | 2.15 | | | | | | | | |
| with | | | T + 2 | 2.52 | 2.17 | 0.20 | 1759 | 159 | | 1659 | 159 | |
| VEGF | | | T + 3 | 1.84 | | | | | | | | |
| Medium | 10 | μg/ml | P1 − 1 | 0.15 | | | | | | | | |

| | | | | | | | | | | |
|---|---|---|---|---|---|---|---|---|---|---|
| with | P1 − 2 | 0.15 | 0.15 | 0.00 | 119 | 3 | ns | 19 | 3 | ns |
| HMWP | P1 − 3 | 0.14 | | | | | | | | |
| sulphated | | | | | | | | | | |
| polysaccharide | | | | | | | | | | |

```
⁽¹⁾Statistical significance threshold
ns: >0.05, not significant
  *: 0.01 to 0.05, significant
  **: 0.001 to 0.01, Very significant
***: <0.001, Extremely significant
sem: standard error of the mean
```

Inter-group comparisons were carried out using the unpaired bilateral Student's t test.

TABLE 6
Effect of the HMWP sulphated polysaccharide according to the invention
on the formation of the pseudotubes—Condition stimulated by the VEGF

| | | | Basic data | | | | | | | | |
|---|---|---|---|---|---|---|---|---|---|---|---|
| | | | Surface of | | | | | | | | |
| | | | the | Average | | % | | | Standardized data | | |
| Treatment | | | pseudotubes | Surface | sem | Stimulated | sem | | % | sem | |
| Compounds tested | | | (mm²) | (mm²) | (mm²) | control | (%) | p⁽¹⁾ | inhibition | (%) | p⁽¹⁾ |
| Non- | Control | T − 1 | 0.16 | 0.12 | 0.02 | 6 | 1 | *** | 100 | 1 | *** |
| stimulated | | T − 2 | 0.09 | | | | | | | | |
| condition | | T − 3 | 0.12 | | | | | | | | |
| Stimulated | Control | T + 1 | 2.15 | 2.17 | 0.20 | 100 | 9 | | 0 | 10 | |
| conditions: | | T + 2 | 2.52 | | | | | | | | |
| VEGF- | | T + 3 | 1.84 | | | | | | | | |
| 100 ng/ml | Sulphated | P1 + 1 | 0.70 | 0.59 | 0.06 | 27 | 3 | ** | 77 | 3 | ** |
| | polysaccharide | P1 + 2 | 0.54 | | | | | | | | |
| | according to | P1 + 3 | 0.53 | | | | | | | | |
| | the invention | | | | | | | | | | |
| | (10 μg/ml) | | | | | | | | | | |

```
⁽¹⁾Statistical significance threshold
ns: >0.05, not significant
  *: 0.01 to 0.05, Significant
  **: 0.001 to 0.01, Very significant
***: +<0.001, Extremely significant
sem: standard error of the mean
```

Inter-group comparisons were carried out using the unpaired bilateral Student's t test.

In the basal condition, only diffuse and weak labelling could be observed, indicating absence of organization of the endothelial cells. The treatment with the reference VEGF (100 ng/ml) clearly led to an organization of the endothelial cells into pseudotubes.

Tested in basal condition, the HMWP compound, at 10 μg/ml, had no significant effect compared with the non-stimulated control condition and did not therefore induce the formation of pseudotubes in the HMVEC/NHDF co-culture.

Tested in stimulated condition, the HMWP compound, at 10 μg/ml, clearly and significantly inhibited the formation of pseudotubes induced by the VEGF (77% inhibition).

### Example 7

### Complete Transcriptome Analysis of the Effects of the HMWP Sulphated Polysaccharide

Complete transcriptome analysis of the effects of the HMWP sulphated polysaccharide was carried out on normal human epidermal keratinocytes (NHEKs) and on normal human dermal fibroblasts (NHDFs) at two points in time during incubation: 4 hours and 24 hours.

### Compound Tested

Compound tested: HMWP sulphated polysaccharide
Concentrations tested:
   0.1 mg/ml on the NHEKs
   3 mg/ml on the NHDFs

### Cultures and Treatments

The keratinocytes were seeded in 24-well plates and the fibroblasts in 12-well plates, then cultured in culture medium for 48 hours.

The culture medium used is as follows:
Keratinocyte-SFM supplemented with 0.25 ng/ml EGF (epidermal growth factor), 25 μg/ml pituitary extract (PE) and 25 μg/ml gentamycin.

The culture medium was then replaced with test medium and the cells were cultured for another 24 hours. The test medium used is as follows: Keratinocyte-SFM supplemented with 25 μg/ml gentamycin.

The cells were then treated or not treated (control) with the compound to be tested and incubated for 4 or 24 hours. All the experiments were carried out in triplicate (n=3).

At the end of the incubation, the culture supernatants were removed and the cell layers were rinsed with a solution of phosphate-buffered saline (PBS). The plates were immediately frozen dry at −80° C.

### RNA Extraction

Before the extraction, the culture replicates were pooled. The total RNA from each sample was extracted using the NucleoSpin® RNA Plus kit (Macherey-Nagel) according to the protocol recommended by the supplier.

### Differential Expression Analysis

The quantity and quality of the RNAs were evaluated by capillary electrophoresis (Bioanalyzer 2100, Agilent). Synthesis of the biotinylated anti-sense RNAs (aRNA) was carried out using the “GeneChip 3′IVT Express” kit (Affymetrix®). For each biotinylated aRNA sample, an electrophoretic profile was produced (Bioanalyzer 2100, Agilent) before and after fragmentation. The hybridization of the labelled and fragmented aRNAs on the Affymetrix® U219 chip (36,000 transcripts and variants) was carried out on the GeneAtlas™ fluidics Affymetrix® hybridization station for 20 hours at 45° C. The U219 chips were then scanned using the GeneAtlas™ Imaging station (Affymetrix®—resolution 2 Inn) in order to generate the signal intensity data.

### Processing of the Data

The signal intensity data are standardized using the Expression Console software (Affymetrix), based on the RMA algorithm. Quality control of the labelling as well as of the hybridization is then carried out.

### Results Obtained

The results obtained are presented in Table 7.

TABLE 7
Transcriptome analysis of the HMWP and effects on the process of cell
proliferation and angiogenesis

| | | Incubation time: | Incubation time: |
|---|---|---|---|
| Treatments | Effects observed | 4 hours | 24 hours |
| Treatment | Effects on the cell | Inhibition of cell | Inhibition of cell |
| of the | proliferation | proliferation by | proliferation by |
| keratinocytes | process | inhibiting the | inhibiting the |
| (NHEK) with | | expression of the | expression of the |
| HMWP | | genes coding for the | genes coding for the |
| at 0.1 mg/ml | | growth factors and | growth factors and |
| | | the genes involved | the genes involved |
| | | in the regulation of | in the regulation of |
| | | cell proliferation | cell proliferation |
| | Effects on | Inhibition of the | Inhibition of the |
| | angiogenesis | expression of genes | expression of genes |
| | | involved in the | involved in the |
| | | angiogenesis | angiogenesis |
| | | process: JAG1, | process: JAG1, |
| | | VEGFA, CYR61 | VEGFA, CYR61 |
| Treatment | Effects on the cell | Increase in the | Increase in the |
| of fibroblasts | proliferation | expression of the | expression of the |
| (NHDF) | process | genes involved in | genes involved in |
| with HMWP | | cell proliferation | cell proliferation |
| at 3 mg/ml | Effects on | Increase in the | Increase in the |
| | angiogenesis | expression of genes | expression of genes |
| | | involved in the | involved in the |
| | | angiogenesis | angiogenesis |
| | | process: C3, | process: C3, |
| | | MMP14 | MMP14 |

This example shows that the HMWP sulphated polysaccharide is capable of inhibiting the cell proliferation process and angiogenesis at a concentration of 0.1 mg/ml on the keratinocytes, whereas it rather tends to stimulate the cell proliferation process and angiogenesis at a concentration of 3 mg/ml on the fibroblasts.

This makes it a good active ingredient for the prevention and/or treatment of redness.

[symbol]

### Example 8

### Evaluation of the HMWP Sulphated Polysaccharide on the Cell Viability of Normal Human Keratinocytes

Human keratinocytes are seeded in 96-well microplates at a density of 20,000 keratinocytes per well (equivalent to 60,000 cells/cm², then left to adhere/proliferate for 24 hours at 37° C. with 5% CO₂ in complete KSFM medium (containing added antibiotics and growth supplements, Gibco 17005).

The human keratinocytes are treated with the HMWP sulphated polysaccharide to be tested in non-supplemented medium (without growth supplements) for 48 hours and incubated at 37° C. with 5% CO₂. Each concentration of the product is evaluated in triplicate. Two positive controls are used: one of cytotoxicity, 10% dimethylsulphoxide (DMSO—Sigma D4540) and one of proliferation, the complete medium (i.e. with growth supplements).

A cell viability/cytotoxicity test (XTT test) is carried out in order to determine the non-cytotoxic doses. The XTT test is carried out by means of the Cell Proliferation Kit II (XTT) (Sigma/Roche Diagnostics, 11465015001).

After the 48 hours of treatment, the wells are carefully rinsed with a phosphate-buffered saline (PBS, Introvogen). The keratinocytes are then brought into contact with a tetrazolium sodium salt solution (XTT) at 0.3 mg/mL. The plates are incubated at 37° C. with 5% CO₂ in darkness. The tetrazolium sodium salt solution (XTT) is also deposited in wells without cells (medium with or without product) in order to produce blanks.

After incubating for 3 hours, the absorbance is measured at 450 nm with a reference at 650 nm. For each condition, the optical density values (OD, absorbance) are averaged.

The viability of the cells treated is expressed as a percentage with respect to the control (untreated cells):

$$\% viability “ sample ” = \frac{OD “ sample ”}{Average OD “ untreated control ”} \times 100$$

A treatment leading to a reduction in viability, below the threshold value of 80% mitochondrial activity with respect to the control, is considered as cytotoxic for the cells. Conversely, an increase in the value evidences an increase in mitochondrial activity, or even in cell proliferation.

The significance of the results is evaluated by comparison of the values with those obtained for the control condition, by the Student's t-test with the following criteria:

| Criterion of the | | |
|---|---|---|
| p-value | Significance (of the difference | Graphical |
| (Student's t-test) | between the values compared) | notation |
| p > 0.05 | not significant | |
| 0.01 < p ≤ 0.05 | significant (at 95%) | * |
| 0.001 < p ≤ 0.01 | very significant (at 99%) | ** |
| p ≤ 0.001 | very highly significant (at 99.9%) | *** |

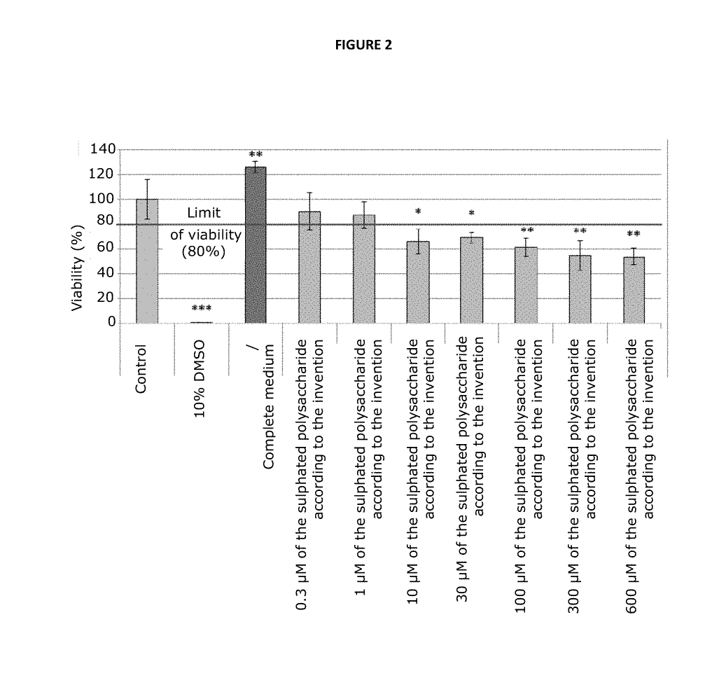

The results obtained are presented in FIG. 2.

### Conclusions

The HMWP sulphated polysaccharide is non-cytotoxic to the keratinocytes after 48 hours of application, at concentrations of 0.3 and 1 μg/mL (above the 80% viability threshold), whereas it appears to be cytotoxic in the strongest doses tested, i.e. from 10 to 600 μg/mL.

### Example 9

### Effects of the HMWP Sulphated Polysaccharide on the Release of VEGF and Interleukin (IL8) by Normal Human Keratinocytes

Human keratinocytes are seeded in 96-well microplates at a density of 20,000 cells/well (i.e. 60,000 cells/cm²), in complete KSFM medium and left to adhere/proliferate at 37° C. under 5% CO₂, 24 hours before treatment.

The HMWP sulphated polysaccharide, tested in 3 non-cytotoxic concentrations (0.1 μg/ml, 0.3 μg/ml and 1 μg/ml), is brought into contact with the keratinocytes for 24 hours at 37° C. with 5% CO₂ (in unsupplemented KSFM medium). The HMWP sulphated polysaccharide is then re-applied for another 24 hours (i.e. 48 hours in total), in the presence or absence of stimulation with IL1β at 20 ng/mL (Bio-Techne/R&Dsystems, 201-LB). Each unstimulated condition is tested in triplicate (n=3 cell wells), and the same applies for each stimulated condition. The assays are then carried out in duplicate.

Positive controls of inhibition of the production of VEGF and of IL8 are used (at 1 μM): EGCG (Epigallocatechin gallate—Tocris 4524) and staurosporine (Sigma S4400) respectively.

### Assay of IL8 and of VEGF

The targets of interest, IL8 and VEGF are assayed by means of kits supplied by Bio-Techne/R&Dsystems (D8000C) and Thermo/Fisher Scientific (EH2VEGF) respectively. The results of the assays are obtained by measuring the absorbance (OD) at a wavelength of 450 nm, with 550 or 570 nm as reference wavelength.

### Assay of the Total Proteins

The total proteins are assayed using the BCA method. The BCA assay kit (Sigma BCA1) is composed of a bicinchoninic acid solution (BCA) (Sigma B9643) and copper sulphate (CuSO₄—Sigma C2284). The standard range is prepared based on BSA (Bovine Serum Albumin—Sigma A9418). The cell pellets are kept dry at −20° C. while awaiting this assay. In order to lyse the cells and alkalinize the reaction medium, the cell pellets are equilibrated at ambient temperature, then placed in alkaline medium for a minimum of 30 minutes. The assay is carried out by adding a mixture of the reagents bicinchoninic acid and CuSO₄. The plate is incubated at 37° C. and the reaction is stopped by placing the plate for a few minutes at 4° C. The assay reading is then carried out at a wavelength of 570 nm.

### Results

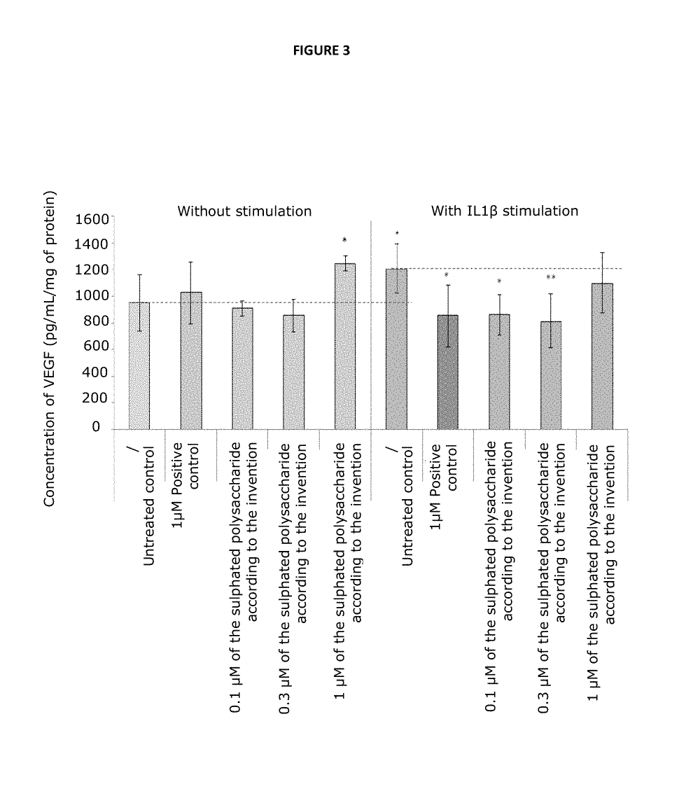

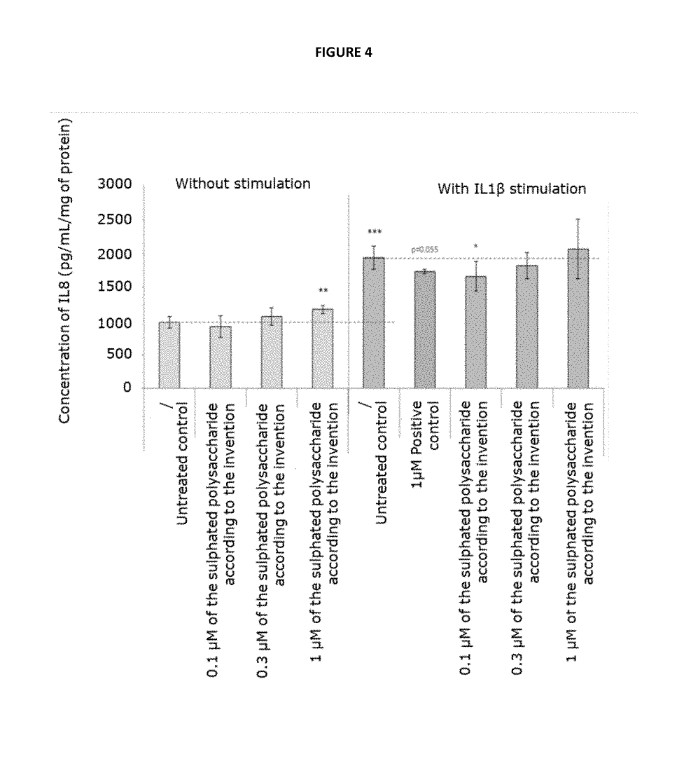

The results obtained by the BCA method are presented in FIGS. 3 and 4.

With the HMWP sulphated polysaccharide, in the case of inflammatory stress, a potential inhibitory effect is observed on the syntheses of VEGF and IL8, at the lowest doses tested.

In fact, following the treatment with the HMWP sulphated polysaccharide, in the two concentrations tested, the lowest of 0.1 and 0.3 μg/mL, a significant reduction in the release of keratinocytes from the VEGF induced by the inflammatory stress (IL1β), of −29% and −33% respectively, with respect to the untreated but stimulated control (significant * or highly significant ** respectively) is obtained. This inhibition is comparable to that obtained with the positive control.

In the strongest concentration tested, 1 μg/mL, the effect of the HMWP sulphated polysaccharide is slight (−9%) and not significant.

As regards IL8, a significant reduction (*) is observed in the release of keratinocytes after induction (inflammatory stress), of −14% after application of 0.1 μg/mL of the product, with respect to the stimulated control. This inhibitory effect is of the same order of magnitude as that recorded with the positive control.

At the dose of 0.3 μg/mL, the effect of the extract is slight (−6%) and not significant and at 1 μg/mL there is no effect.

In the absence of stimulation, there is no significant inhibition of the basal releases of VEGF or of IL8, with any of the three doses tested:
At the doses of 0.1 and 0.3 μg/mL, a slight reduction in the VEGF released is recorded (−5% and −10% respectively, in a non-significant manner). Conversely, at the strongest concentration tested (1 μg/mL,) a stimulation of this production of +31% (significant *) is observed.
At the concentration of 0.1 μg/mL, a slight reduction in IL8 is recorded (−7%, in a non-significant manner). Conversely, at 0.3 and 1 μg/mL, a slight stimulation of this production appears (+8% and +19% respectively, in a non-significant or significant manner **).

### Conclusions

The HMWP sulphated polysaccharide makes it possible:
at the doses of 0.1 and 0.3 μg/mL, to counter the release of the VEGF in an inflammatory stress context,
at the dose of 0.1 μg/mL, to also combat the production of IL8 (under inflammatory stress).

1. Sulphated polysaccharide extracted from a red alga of the Haliptilon subulatum species or a salt thereof for its use in the prevention and/or treatment of inflammatory diseases in humans or animals.

2. Sulphated polysaccharide according to claim 1, wherein said sulphated polysaccharide is a VEGF antagonist.

3. Sulphated polysaccharide according to claim 1, wherein the sulphated polysaccharide has a molecular weight less than or equal to 500 kDa.

4. Sulphated polysaccharide according to claim 1, wherein the sulphated polysaccharide has a molecular weight comprised between 10 kDa and 250 kDa.

5. Composition comprising the sulphated polysaccharide according to claim 1 and at least one pharmaceutically or dermatologically or cosmetically acceptable excipient.

6. Composition according to claim 1, further comprising at least one other active agent, the active agent being in particular selected from anti-redness agents, decongestants, antibacterial agents, antiseptics and antimicrobials, anti-inflammatories, anti-irritant and/or soothing agents, cicatrizants and/or restructurants of the skin barrier, antioxidants, moisturizing and/or emollient agents, anti-aging agents, mineral or organic sun filters and screens and sun protection active agents.

7. Composition according to claim 5, suitable to be administered by oral, topical or injectable route or in the form of supplements and/or food products.

8. Composition according to claim 5, wherein the composition is a cosmetic composition or a dermatological composition.

9. Dermatological composition according to claim 8 for its use in the prevention and/or treatment of inflammatory diseases, in particular of the skin.

10. Dermatological composition according to claim 9, wherein the inflammatory disease is selected from the group comprising: psoriasis, atopical dermatitis, rosacea, couperose, acne, vulgar warts, bullous skin diseases, contact eczema, skin cancers, redness, erythema, telangiectasia, inflammations of the skin associated with UV exposure.

11. Sulphated polysaccharide according to claim 2, wherein the sulphated polysaccharide has a molecular weight less than or equal to 500 kDa.

12. Sulphated polysaccharide according to claim 2, wherein the sulphated polysaccharide has a molecular weight comprised between 10 kDa and 250 kDa.

13. Sulphated polysaccharide according to claim 3, wherein the sulphated polysaccharide has a molecular weight comprised between 10 kDa and 250 kDa.

14. Composition comprising the sulphated polysaccharide according to claim 2 and at least one pharmaceutically or dermatologically or cosmetically acceptable excipient.

15. Composition comprising the sulphated polysaccharide according to claim 3 and at least one pharmaceutically or dermatologically or cosmetically acceptable excipient.

16. Composition comprising the sulphated polysaccharide according to claim 4 and at least one pharmaceutically or dermatologically or cosmetically acceptable excipient.

17. Composition according to claim 2, further comprising at least one other active agent, the active agent being in particular selected from anti-redness agents, decongestants, antibacterial agents, antiseptics and antimicrobials, anti-inflammatories, anti-irritant and/or soothing agents, cicatrizants and/or restructurants of the skin barrier, antioxidants, moisturizing and/or emollient agents, anti-aging agents, mineral or organic sun filters and screens and sun protection active agents.

18. Composition according to claim 3, further comprising at least one other active agent, the active agent being in particular selected from anti-redness agents, decongestants, antibacterial agents, antiseptics and antimicrobials, anti-inflammatories, anti-irritant and/or soothing agents, cicatrizants and/or restructurants of the skin barrier, antioxidants, moisturizing and/or emollient agents, anti-aging agents, mineral or organic sun filters and screens and sun protection active agents.

19. Composition according to claim 4, further comprising at least one other active agent, the active agent being in particular selected from anti-redness agents, decongestants, antibacterial agents, antiseptics and antimicrobials, anti-inflammatories, anti-irritant and/or soothing agents, cicatrizants and/or restructurants of the skin barrier, antioxidants, moisturizing and/or emollient agents, anti-aging agents, mineral or organic sun filters and screens and sun protection active agents.

20. The dermatological composition of claim 10, wherein the UV exposure comprises photoirritation, photosensitization, photoaging, photocarcinogenesis, venous lymphatic insufficiency, or heavy legs syndrome.
